(19) United States
(12) Patent Application Publication
(10) Pub. No.: US 2025/0325362 A1
(43) Pub. Date: Oct. 23, 2025

(54) OPTIMIZATION OF HIGH DEFINITION AND EXTENDED DEPTH OF FIELD INTRAOCULAR LENS

(71) Applicant: Z Optics, Inc., Cookeville, TN (US)

(72) Inventor: Matthew Schuster, Cookeville, TN (US)

(21) Appl. No.: 18/854,889

(22) PCT Filed: Apr. 13, 2023
(86) PCT No.: PCT/US2023/018533

§ 371 Date: Oct. 7, 2024

Related U.S. Application Data
(60) Provisional application No. 63/330,590, filed on Apr. 13, 2022.
(60) Provisional application No. 63/340,617, filed on May 11, 2022.
(60) Provisional application No. 63/425,587, filed on Nov. 15, 2022.

(51) Int. Cl. A61F 2/16

(52) U.S. Cl. CPC A61F 2/164

(57) ABSTRACT

Disclosed is a configuration for a lens optimized, enhanced or improved for a desired extended depth of focus, extended depth of field or other characteristic(s) of the lens. The disclosed systems and methods enable a choice of a lens parameter, such as an optical zone diameter, radius of curvature, conic constant and alpha coefficients, to maximize or otherwise enhance one or more characteristics of the lens.

### CROSS-REFERENCE TO RELATED APPLICATIONS

This application claims priority to U.S. Patent Application Nos. 63/330,590, filed on Apr. 13, 2022, 63/340,617, filed on May 11, 2022, and 63/425,587, filed on Nov. 15, 2022, entitled “OPTIMIZATION OF HIGH DEFINITION AND EXTENDED DEPTH OF FIELD INTRAOCULAR LENS”, the contents of which are hereby incorporated by reference herein in their entirety.

### BACKGROUND

Conventional optical lens solutions for extending depth of focus while eliminating common drawbacks include diffractive and refractive multifocal lenses having discrete zones. Other conventional solutions include aspheric lenses with a power profile that varies over the radius of the lens. In the case of the aspheric lens shape, the degree to which the power varies over the radius is set by varying a conic constant K and the nth order aspheric coefficient terms.

Such techniques have drawbacks. For example, in the case of an intraocular lens (IOL), the placement of discrete zones is sensitive to pupil size. If a particular power zone is either exposed by a dilated pupil or covered by a constricted pupil, it will dramatically change the resultant power of the IOL. In another example, multi-zone configurations by definition introduce unwanted visual artifacts such as halos. For an object at a given distance, one zone might be in excellent or good focus while the other zones may be at somewhat less than perfect focus. This introduces multiple images and halos around the primary object. Further, with a small number of zones, the halos become more pronounced and distinct.

There is a need for lens configurations that alleviate or eliminate the aforementioned drawbacks.

### SUMMARY

Disclosed is a configuration solution for an IOL that is optimized, enhanced or improved for a desired extended depth of focus, extended depth of field or other characteristic(s) of the IOL. The disclosed systems and methods enable a choice of a lens parameter, such as an optical zone diameter, radius of curvature, conic constant and alpha coefficients, to maximize or otherwise enhance the desired characteristics. Some example characteristics that may be optimized or enhanced are depth of focus, minimizing poor (sub 20/20) performance zones, and insuring robust behavior with regard to de-centration, tilt, off axis light sources, surgical implantation error, and errors in choice of IOL Power.

The details of one or more variations of the subject matter described herein are set forth in the accompanying drawings and the description below. Other features and advantages of the subject matter described herein will be apparent from the description and drawings, and from the claims.

### BRIEF DESCRIPTION OF THE DRAWINGS

FIGS. 1A and 1B illustrates a basic method of reducing monochromatic aberrations and increasing or extending depth of field using pupil size for a near-sighted eye.

FIGS. 2A and 2B illustrate a basic method of reducing monochromatic aberrations and increasing depth of field using pupil size for a far-sighted eye.

FIGS. 3A and 3B illustrate a basic method of reducing monochromatic aberrations and increasing depth of field using pupil size for an emmetropic eye.

FIGS. 4A and 4B illustrate the basic method of reducing chromatic aberrations using pupil size.

FIGS. 5A and 5B illustrate the basic concept of the virtual aperture to limit the effective pupil size.

FIGS. 6A, 6B, and 6C illustrates an overall structure of an example IOL.

FIG. 6D shows another embodiment of an example IOL.

FIG. 7 illustrates an example IOL with hexagonal sampled micro-lenses in a virtual aperture zone.

FIG. 8 Illustrates an example partition of an optical zone of an IOL to provide both near and distance vision partitions.

FIGS. 9-11 relate to systems for modifying a subsurface region of an IOL.

FIG. 12 shows a computing system.

### DETAILED DESCRIPTION

Before the present subject matter is further described, it is to be understood that this subject matter described herein is not limited to particular embodiments described, as such may of course vary. It is also to be understood that the terminology used herein is for the purpose of describing particular embodiments only, and is not intended to be limiting. Unless defined otherwise, all technical terms used herein have the same meaning as commonly understood by one skilled in the art to which this subject matter belongs.

Disclosed is a configuration solution for an IOL that is optimized, enhanced or improved for desired characteristic(s) of the IOL. The disclosed systems and methods enable a choice of a lens parameter (or parameter of an equation that defines the lens), such as optical zone diameter, radius of curvature, conic constant, alpha coefficients, and/or polynomial coefficient, to maximize or otherwise enhance the desired characteristics. Some example characteristics that may be optimized or enhanced are depth of focus, minimizing poor (sub 20/20) performance zones, and insuring robust behavior with regard to de-centration, tilt, off axis light sources, surgical implantation error, and errors in choice of IOL Power.

Further disclosed are systems, devices, and methods that overcome limitations of IOLs at least by providing a phakic or aphakic IOL that provides correction of defocus and astigmatism, decreases higher-order monochromatic and chromatic aberrations, and provides an extended depth of field to improve vision quality. The disclosed IOL is sometimes referred to herein as the Z+ optic or Z+ IOL. U.S. Pat. No. 10,285,807, U.S. patent application Ser. No. 16/380,622 and PCT application PCT/US20/37014 describe related systems and methods and are incorporated herein by reference in their entirety.

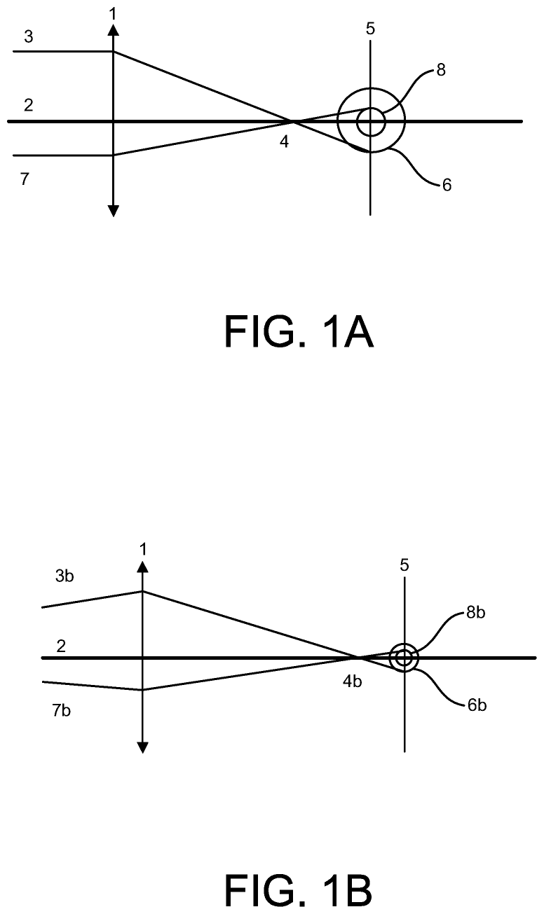

A description of the basic principle used to reduce monochromatic and chromatic aberrations and provide an increased depth of field is now provided. FIG. 1A schematically illustrates a single converging lens 1 centered on an optical axis 2. An incident ray 3 from a distant object is parallel to the optical axis and intersects the focal point 4 (with a suffix b, c, d, e, or f based on the corresponding figure) of the lens. If the lens power is properly selected, the focal point coincides with the observation plane 5, otherwise there is a mismatch between the lens power and the location of the observation plane such that the focus is in front of or behind the observation plane.

In FIG. 1A, the focal point is in front of the observation plane. If all incident rays are traced with the same ray height as incident ray 3, a blur circle 6 is located on the observation plane 5. The observation plane is oriented orthogonal to the optical axis and so is shown as a vertical line in the figure. The blur circles 6 and 8 are shown in the plane of the figure for visualization convenience, however, the blur circles are actually contained in the observation plane. Other parallel incident rays with ray height less than incident ray 3 fall inside this blur circle 6. One such ray is parallel incident ray 7 which is closer to the optical axis than incident ray 3. Incident ray 7 also intersects the focal point 4 and then the observation plane 5. Tracing all incident rays with ray height equal to incident ray 7 traces out blur circle 8 which has a diameter smaller than that of blur circle 6.

FIG. 1B illustrates the same optical system in FIG. 1A, but now the incident rays are for an object closer to the optical system as indicated by the slopes on incident rays 3b and 7b. The effect is that the focus point 4 (with a suffice a, b, c, d, or f based on the corresponding figure) for the closer object is now closer to the observation plane and both of the blur circles 6b and 8b are smaller than their counter parts in FIG. 1A, but the principle is the same: rays which intersect the lens 1 closer to the optical axis have smaller blur on the observation plane. To relate this simple optical construction of FIG. 1 to the human eye, the converging lens 1 represents the principal plane of the eye's optics including the cornea and the crystalline lens or an intraocular lens. Observation plane 5 represents the retina. As drawn the focal point 4 is in front of the observation plane (retina), so this figure is for a myopic or near-sighted eye. The size of the blur circles 6 and 8 (or 6b and 8b) represents the amount of defocus on the retina, where a smaller blur circle diameter provides clearer vision than a larger blur circle diameter.

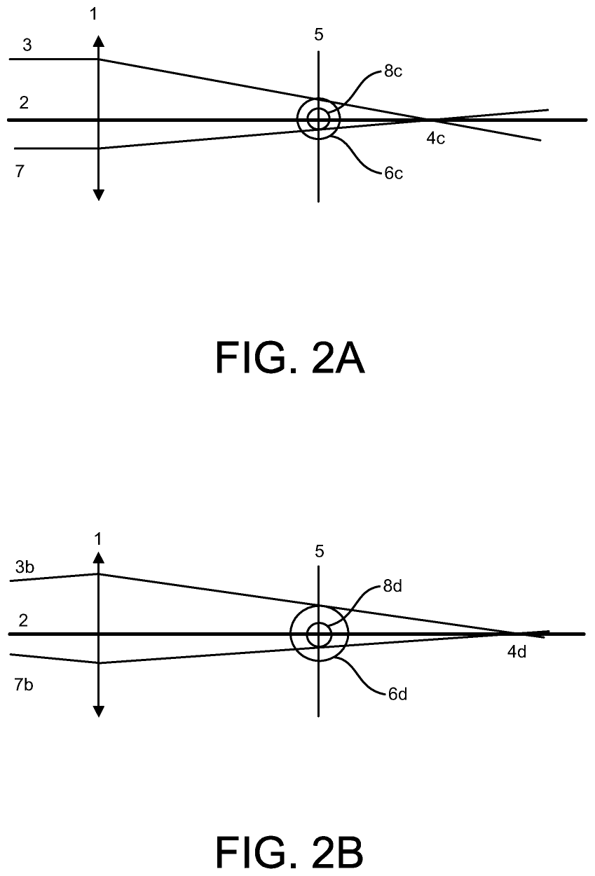

Note that the same relationship regarding incident ray height and blur circle size also holds for hyperopic or far-sighted eyes. This is schematically illustrated in FIGS. 2A and 2B, which show rays corresponding to a far-sighted eye. In FIG. 2A for rays 3 and 7 from a distant object and in FIG. 2B for rays 3b and 7b, smaller ray height leads to a smaller blur circle on the retina (observation plane).

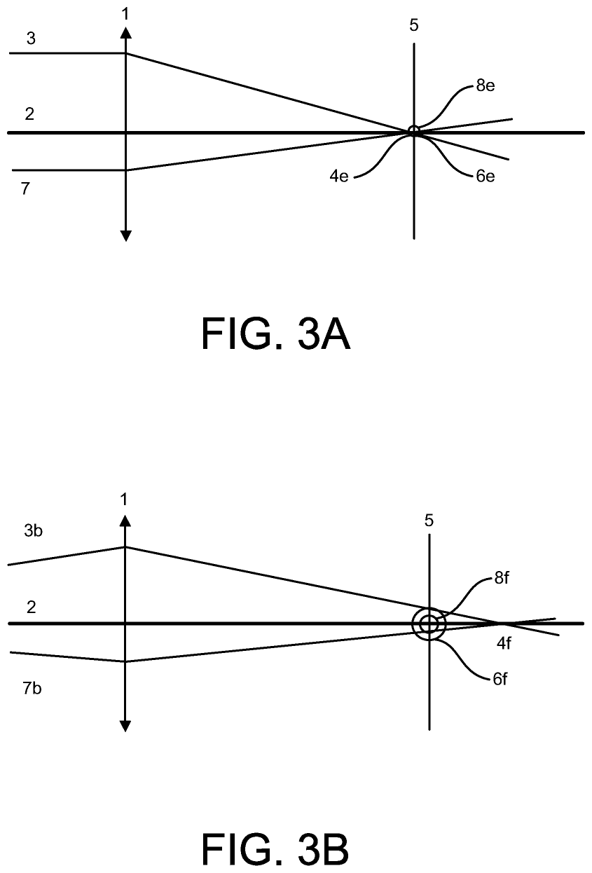

Similarly, FIGS. 3A and 3B (collectively referred to as FIG. 3) show that the same parallel ray height to blur circle diameter property holds for an emmetropic eye. For a distant object, the focal point 4e is now at the retina (since the eye is emmetropic) and the blur circles 6e and 8e have zero radius. For a closer object, the focal point 4f is behind the retina and blur circle 8f corresponding to ray 7b which is closer to the optic axis has a smaller diameter than blur circle 6f corresponding to ray 3b which is further from the optic axis.

In general, an eye has aberrations, which means that as an incident ray location changes, the focal point in the eye also changes. But regardless of where the focal points are located (in front of-, on-, or behind the retina), as incident ray heights are reduced so are the blur circle diameters on the retina. Stated another way, for a given amount of defocus (dioptric error) in the eye, vision is improved as the height of incident rays is reduced. This principle is used when someone squints causing the eyelids to block the incident rays further from the optic axis of the eye in an attempt to see an out-of-focus distant or near object more clearly.

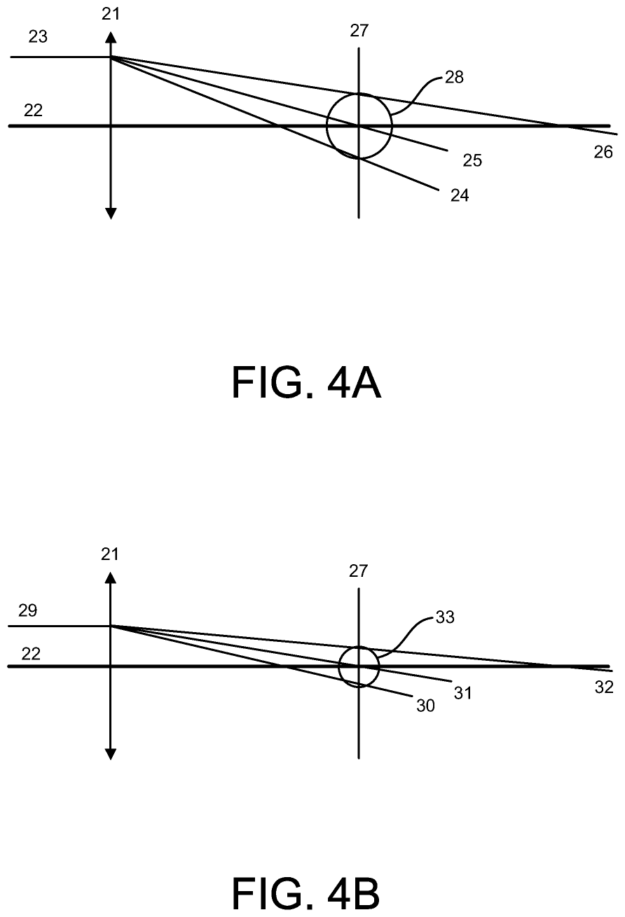

The ray tracing illustrated in FIGS. 1A-3B is for a single wavelength of incident light. For polychromatic light, multiple wavelengths are present. This is commonly illustrated by three rays of different wavelengths as shown in FIGS. 4A and 4B (collectively referred to as FIG. 4). It is well known that for the components of the eye and typical optical materials, as the wavelength of light increases, the refractive index decreases.

In FIG. 4A, a converging lens 21 has optical axis 22. An incident chromatic ray 23 consists of three wavelengths for blue (450 nm), green (550 nm), and red (650 nm) light which approximately span the range of visible light. Due to different indices of refraction for the three wavelengths, the blue light ray 24 is refracted more than the green light ray 25, and the green light ray is refracted more than the red light ray 26. If the green light ray is in focus, then it crosses the observation plane 27 at the optical axis. The chromatic spread of these three rays lead to a chromatic blur 28 on the observation plane.

In FIG. 4B, the incident chromatic ray 29 has a lower ray height than the chromatic ray 23 in 4A. This leads to smaller chromatic blur 33 at the observation plane. Thus, just as for the monochromatic blur of FIGS. 1A-3B, chromatic blur is decreased as the chromatic ray height is decreased. The situation in FIG. 4 can be related to the eye by considering converging lens 21 to be the principal plane of the eye and observation plane 27 to be the retina. The human eye normally has a large amount of chromatic aberration (about 1.0 to 1.2 diopters over the central visual range) so this reduction in chromatic aberration can be significant leading to a noticeable improvement in the eye's visual quality, especially as measured by its contrast sensitivity.

Taken together, FIGS. 1A-4B illustrate that decreasing ray height decreases both monochromatic and chromatic aberrations at the retina, thus increasing the quality of vision. This can be accomplished by either blocking rays with larger distance from the optical axis by decreasing the pupil diameter or by spreading light from these rays evenly and/or widely across the retina so that more aberrant rays contribute much less light to the central retinal blur circle. Another feature of this effect is that the depth of field is increased as the ray height is decreased as illustrated in FIGS. 1B, 2B, and 3B.

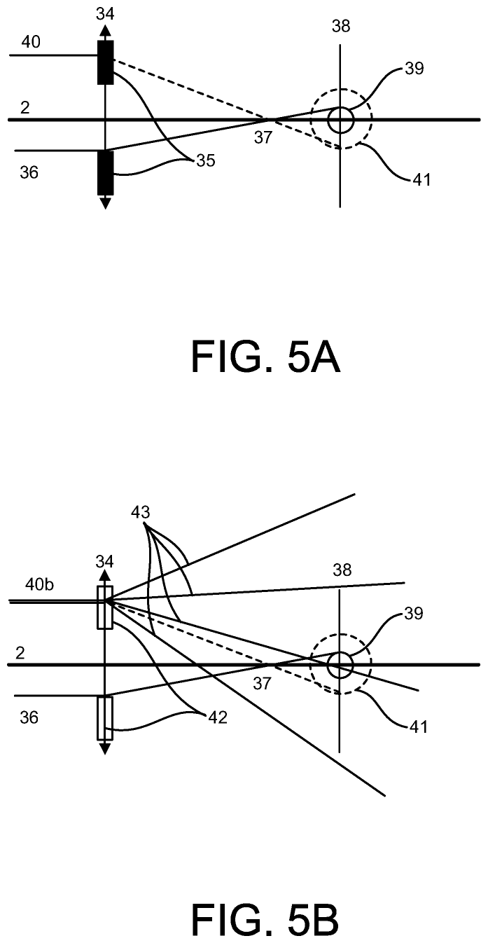

FIG. 5A shows a converging lens 34 with optical axis 2 and aperture 35. Incident parallel ray 36 just clears the aperture and thus passes through the lens focal point 37 and intersects the observation plane 38. All parallel rays with the same height as ray 36 trace a small blur circle 39 on the observation plane. Incident parallel ray 40 is blocked by the aperture, and thus it cannot continue to the observation plane to cause a larger blur circle 41. In this way, an aperture which reduces the incident ray height reduces the blur diameter on the observation plane.

FIG. 5B illustrates a “virtual aperture”. That is, it is not really an aperture that blocks rays, but the optical effect is nearly the same on central vision. In this figure, bundle of rays 40b incident on the virtual aperture propagate through the virtual aperture 42 and through refraction, diffraction, scattering, reflection, and/or diffusion yield rays 43 which are widely spread out so there is very little contribution to stray light (blurring light) at any one spot on the observation plane. This is a principal mechanism of operation of the disclosed IOL. The virtual aperture can be achieved via a surface modification, subsurface modification, or structure added to or positioned relative to the IOL, such as a mask structure. For example, the mask structure can be a ring shaped structure or any ring-shaped mask that occludes at least a portion of light from passing through the IOL.

### Exemplary Optical Layout of the IOL

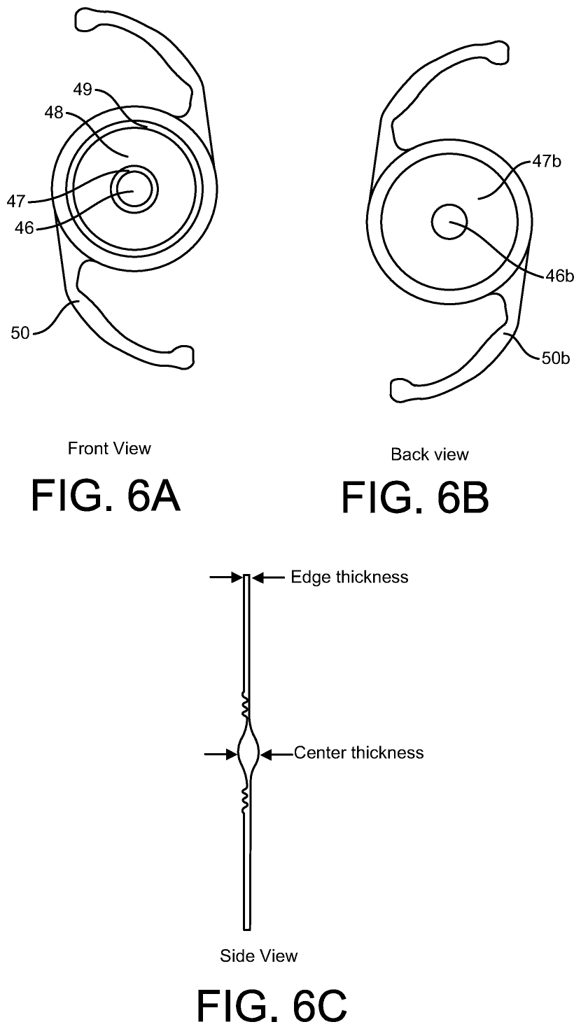

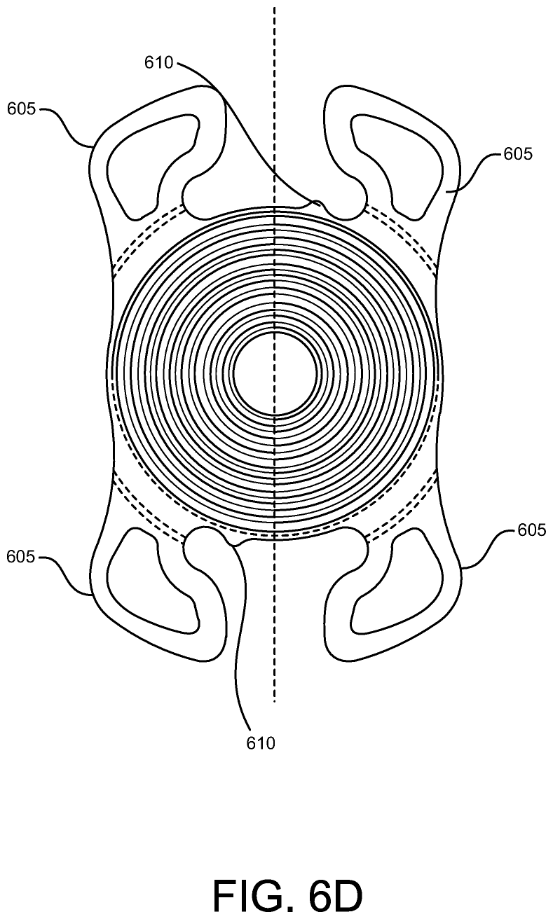

FIGS. 6A-6C illustrate a layout of an example IOL that employs optical principles to achieve the benefits of decreased monochromatic and chromatic aberrations and increased depth of field. FIG. 6A shows a front view of the IOL wherein the front view may be an anterior view. FIG. 6B shows a back view of the IOL wherein the back view may be a posterior view. FIG. 6C shows a side view of the IOL. The IOL includes a central optical zone 46 (with back side 46b) that provides correction of defocus, astigmatism, and any other correction required of the lens such as spherical aberration. Generally, for an IOL using a virtual aperture, the central optical zone diameter is smaller than that of a traditional IOL. This leads to a smaller central thickness which in turn makes the IOL easier to implant and allows a smaller corneal incision during surgery, such as an incision on the order of 2.2 mm. The central optical zone can achieve variable transmissivity of light.

The IOL includes a virtual aperture 48 that is positioned further peripherally outward relative to the center location of the central optical zone 46. Moving peripherally outward from the virtual aperture 48, at least one IOL haptic 50 (with back side 50b) is located on the IOL. The haptic 50 can be formed of one or more arms that extend peripherally outward to define a peripheral most edge of the IOL. In an example, the optical zone has a diameter of 1.5 mm. The haptic 50 may define an outermost peripheral region of the IOL. A first plurality of light rays incident on an anterior optical surface of the optical zone can pass through the optical zone to form an image on a retina when the IOL is positioned in an eye, while a second plurality of light rays incident on an anterior virtual aperture surface are dispersed widely downstream from the IOL towards and across the retina, such that the image comprises an extended depth-of-field and further wherein the virtual aperture reduces monochromatic and chromatic aberrations in the image. The optical zone can comprise at least one of bifocal optics, trifocal optics and multifocal optics.

The virtual aperture is connected to the optical zone 46 by an optional first transition region 47, which is located at a peripheral edge of the optical zone 46 such that the virtual aperture is a first periphery region that surrounds or partially surrounds the optical zone. The haptic can comprise a second periphery region for positioning the intraocular lens within an eye. The first transition region is located peripherally outward of the optical zone 46. An optional second transition region 49 connects the haptic 50 to the virtual aperture 48. The first transition region 47 and the second transition region 49 are configured to ensure zero- and first-order continuity of an outer surface of the IOL on either side of the respective transition region. A common way to implement these transition regions is a polynomial function such as a cubic Bezier function. Transition methods such as these are known to those skilled in the art. On the back side of the IOL is a central optic zone 46b, a haptic 50b, and a transition 47b between them. FIGS. 6A-6C are not necessarily to scale, and the haptic shape is for illustration purposes only. Other haptic shapes and sizes known to those skilled in the art would be suitable as well. The first and second transition regions are not necessarily present per se in the IOL.

The IOL has an anterior surface and a posterior surface and the components of the IOL including the optical zone 46, the first transition region 47, the second transition region 49, the virtual aperture 48, the haptic 50 can each have a respective anterior surface and posterior surface. The optical zone 46 has an anterior optical surface that can include at least one multifocal zone and/or a toric region. At least a portion or region of the anterior surface and/or the posterior surface, such as in the region of the virtual aperture or other portion of the IOL, can have a surface contour or shape that achieves a desired or predetermined effect for light passing therethrough. In nonlimiting examples, the surface contour of the anterior surface and/or the posterior surface includes a region with a ripple-type contour such as a wave shape or an undulating shape that forms a series of raised and lowered surfaces. The surface contours can achieve various effects with respect to light passing through the IOL. For example, the surface contour can achieve a wide or wider spread of stray light depending upon the type of surface contour used. The surface contour can be used to achieve a spread of stray light which is guided away from a focal point of the retina.

FIG. 6D shows a front view of another embodiment of an IOL, which includes a central optical zone, a plurality of peripheral haptics 605, and at least one zone having a surface contour such as a ripple or wave as described further below. In an example, the optical zone has a diameter of 1.5 mm and serves as a lens which brings distant objects into sharp focus on the central retina.

The IOL includes one or more orientation structures 610 such as one or more protrusions or nubs. In the illustrated embodiment, the orientation structures 610 are positioned on a peripheral edge of a portion of the IOL with at least one orientation structure 610 on the first side of a vertical meridian of the IOL and a second orientation structure 610 on a second side of the vertical meridian. Meridian. The vertical meridian is shown as a dashed line in FIG. 6D. The orientation structures 610 are configured to allow a clinician, such as a surgeon, to easily detect that the IOL has a correct side facing the front of the eye. Note that if the IOL were oriented with the back side facing the front of the eye, the orientation structures 610 would be counter-clockwise with respect to the vertical of the lens.

A discussed, the haptic(s) 605 provide a mechanical interface with the eye and holds the various zones of the IOL at its proper position relative to the eye.

### Example Optic Zone Details—Hexagonal Micro-Lens Virtual Aperture

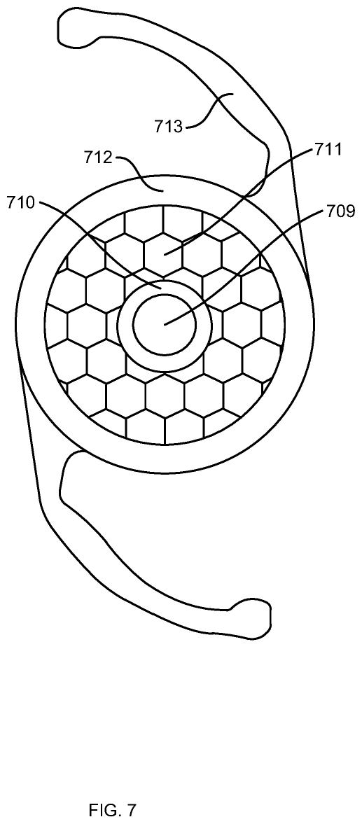

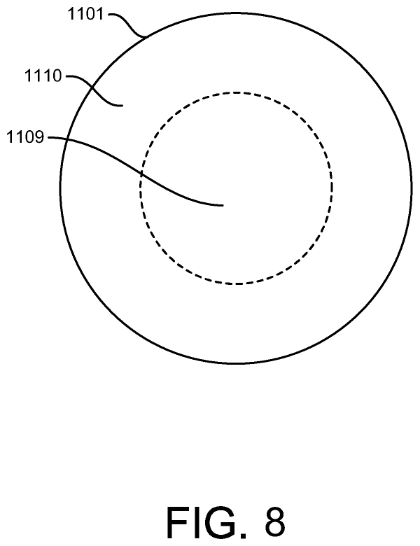

FIG. 7 illustrates a front view of an IOL that includes a virtual aperture having one or more hexagonal structures. The IOL has central optical zone 709, a first transition zone 710, a hexagonal micro-lens virtual aperture 711, a second transition zone 712, and a haptic 713. The first transition zone 710 connects the central optical zone 709 to the hexagonal micro-lens virtual aperture 711 while the second transition zone 712 connects the hexagonal micro-lens virtual aperture 711 to the haptics 713.

The virtual aperture employs a two-dimensional hexagonal sampled array of micro-lenses which mimics the photo sensor sampling of the retina. This arrangement is a beneficial layout for widely spreading light across the retina when the IOL is implanted in an eye.

The hexagonal micro-lens virtual aperture 711 include a plurality of hexagonal shaped microstructures positioned on a front side and/or a backside of the IOL. The hexagonal shape is with respect to an outer boundary of each hexagonal micro-structure has an outer boundary defined by a hexagon microstructure when viewed from a front or rear of the IOL. That is, a hexagonal micro-structure can have an outer boundary defined by a hexagon. A small lens is placed inside the bounds of each of the hexagonal micro-structures. The lens can be a structure that is positioned on or in the micro-structure. The lens may also be monolithically formed as part of the microstructure during manufacture. To help prevent unwanted patterning of light on the retina, the centers of micro-lenses inside each hexagon are randomly moved or positioned on the IOL, and the radii of the micro-lenses are also adjusted. To facilitate manufacturing of the hexagonal micro-lens virtual aperture, between the hexagon boundaries of the micro-lenses, a blending region or fillet is placed with a radius of curvature greater than the radius of a lathe cutter that forms the micro-lens. This radius is on the order of 0.05 mm in a non-limiting example.

The hexagon can have a variety of dimensions. In an embodiment, the hexagon of a micro-structure is more tall than wide. In another embodiment, the hexagon of a micro-structure is more wide than tall. In another embodiment, the outer boundary of a micro-structure is an arbitrarily-shaped polygon.

With reference still to FIG. 7, the first transition zone 710 is configured to provide a smooth structural blend between the edge of the optical zone 709 and the central hexagonal micro-lens region 711. The second transition zone 712 is responsible for providing a smooth structural blend between the peripheral hexagonal micro-lens region 711 and the haptic 713. These transition regions can be effectively accomplished using Bezier curves or portions of Bezier surfaces to define a surface of the respective zone. Other transition functions can be suitable as well and are known to those skilled in the art. It should be appreciated that any of the embodiments of the IOLs described herein can be configured to not include any transition zones. In an embodiment, the system does not have a first transition zone 710 or a second transition zone. In another embodiment, the system has only one of a first transition zone or a second transition zone.

The micro-lenses are implemented as one or more outer surfaces defined at least partially by a sphere, conicoid, or other similar outer surface that can achieve high optical power to widely spread incoming light rays across the retina. For example, the micro-lenses are implemented as one or more outer surfaces defined at least partially by a prismatic or pyramid shape. As an example, in the following discussion there are illustrated embodiments with spherical micro-lenses.

### Multi-Region Optical Zone

FIG. 8 schematically illustrates a multi-region, such as two-region, optical zone 1101 that can be included in any IOL described herein. The regions are indicated 1109 and 1110. These represent two distinct regions in the optical zone for two distinct powers. For example, a first discrete region is a central region 1109 is normally for providing distance vision. A second discrete region is a peripheral region 1110 is normally for providing near vision. The “add” of the near vision region is around 3.0D and in the range of 2.0 to 3.5D.

Due to the special nature of IOL's optical mechanism of action, providing a bifocal optical zone is not as problematic as normal size optical zones of 5.0 mm and larger. This is because the extra aberrations caused by incident rays which are outside the central optical zone diameter of, typically, 1.5 mm, are widely distributed across the retina so as not to negatively affect the central vision of the eye.

In an example configuration, the distance power region of the central optic takes up 75% of the optic zone area and the near power region of the central optic takes up 25% of the optic zone area. Since the diameter of the central optic zone is typically 1.5 mm, the central region 1109 of the optical zone has diameter 1.3 mm and the remainder of the optic zone provides 25% for the near vision region 1110.

For some eyes it can be preferred to have the distribution of distance region area and near region area portioned to 50% each or 25% for distance and 75% for near vision. Providing one eye with a majority of the optic zone area for distance vision, such as 75 to 100%, and the other eye with more area optical zone area for near vision may would be used for extended depth of focus/monovision patients. In this case, both eyes have extended depth of focus, but one eye (usually the dominant eye) has slightly better performance for distance vision and the other eye has slightly better visual performance for near vision.

To provide the desired optical powers for the optic zone regions, either conic refractive profiles can be used, or diffractive profiles can be used.

In the case of simple conic refractive profiles, each optic zone provides its optical power via a conic curve such that the apical radius of curvature provides the desired optical power and the conicity (K) value is set to reduce spherical aberrations for the region. Optimization to find the apical radius and the conicity can be done numerically using commercially available optical design programs such as Zemax or using closed form analytical equations. Both of these methods are known to those skilled in the art. Additionally, the conicity value can be adjusted to further enhance the depth of field performance of the IOL. Conicity values in the range of −7.5 to −9.5 and typically, −8.717 provide such an enhancement for a equal biconvex conic optic zone.

When simple conic refractive profiles are used and the central region 9 of the optical zone provides distance vision and the peripheral region 10 provides near vision, the transition between the regions is negligibly small. This is the preferred arrangement as transition regions generally cause stray light that would otherwise be properly focused by one of the two optical power regions.

When simple conic refractive profiles are used and the central region 9 of the optical zone provides near vision and the peripheral region 10 provides distance vision, the transition between the regions is required to smoothly join the regions. This transition profile is generally implemented by either a Bezier curve or a circular fillet, both of which are known to those skilled in the art.

### Subsurface Modifications of IOL

In an embodiment, at least one region of the IOL, such as the virtual aperture 48 of the IOL, includes at least one subsurface modification comprising a modification to at least a portion of the internal structure of the IOL. The IOL can include such a subsurface modification as well as an optional external surface feature (such as a shape change or contour on the external surface) on an anterior and/or posterior external surface of the IOL. The subsurface modification is configured to achieve a desired optical effect on light that passes therethrough or otherwise interacts with the subsurface modification, such as to diffuse light, homogenize light, or redirect light for example. The subsurface modification of the IOL provides an alternate, efficient, and repeatable mechanism for at least one region of the IOL to diffuse and homogenize light passing therethrough. A degree or level of diffusion and homogenization can be tailored to specific requirements by varying the size of laser damage spots or a modified refractive index loci as described below. The spacing or density of the placement of the damage spots or loci can be varied as can a quantity of layers of such damage spots or loci to achieve a desired level of light diffusion. The configuration of the damage spots or loci can also be used to achieve directional control of light such as to steer light in a desired direction. This enables fine tuning and customization of the optical properties of the IOL or of a light diffuser device.

In an embodiment, the subsurface modification(s) are not positioned in the virtual aperture but are rather part of an optical correction zone of the IOL, which may or may not be in the virtual aperture 48 region of the IOL. In another embodiment, the subsurface modifications form a light diffusion region of an IOL or of a light transmitting body or structure that is not an IOL. For example, the features described herein can be used in a light diffuser device that is not an IOL.

In a first example embodiment of a subsurface modification, a laser is configured to interact with an internal region (i.e., a subsurface region or location) of the IOL to achieve the subsurface modification, such as a modification to the structure of the IOL at the subsurface location. The subsurface region is positioned between at least an anterior surface and a posterior surface of the IOL. In an example, a laser is focused below the surface of the IOL such as to heat the material of the IOL and form a damage region or damage spot located within the material of the IOL at a subsurface location.

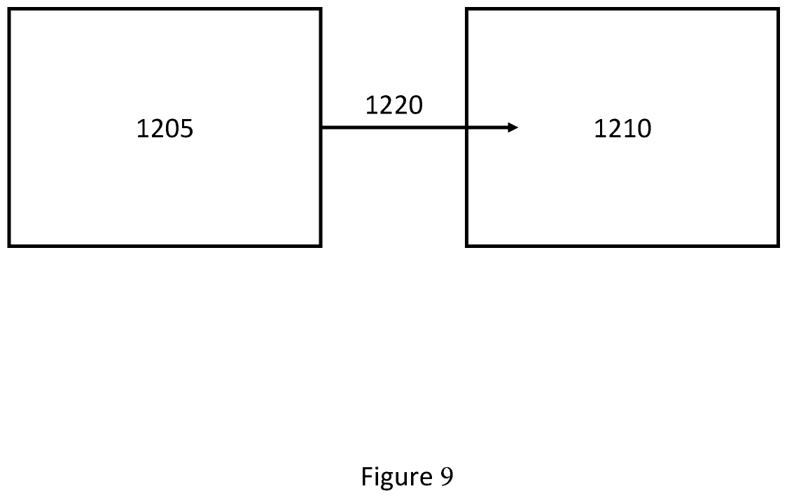

FIG. 9 shows a schematic representation of a laser system 1205 that is configured to interact with an IOL 1210 (or with a piece or body of material that is subsequently formed into or otherwise incorporated into the IOL 1210 or that forms a device that is not an IOL such as a light diffuser device.) The laser system 1205 is configured to emit a laser 1220 that interacts with the IOL, such as laser 1220 that focuses or otherwise emits a predetermined amount of energy at a subsurface location of the IOL 1210.

The laser system 1205 is configured to emit the laser 1220 such that the laser 1220 is focused below the surface of the IOL material (such as a glass or polymer material in a non-limiting example) or that is configured to emit a predetermined level of energy at a subsurface location. In an embodiment, the laser is pulsed at a high rate. The laser 1220 creates one or more microscopic damage points inside (i.e. below an external surface of or between an anterior surface and posterior surface of) the IOL material. In an example embodiment, the pulsed laser causes rapid material heating and expansion in a vicinity of the focused laser spot, which create stresses and small-scale fracturing and gas expansion of the material to thereby form a damage spot. The resultant fracture or damage spot can have extremely small dimension (such as on the order of 10s of microns).

The laser can be moved rapidly and accurately in a lateral X/Y direction while focused at a particular depth in the material relative to an anterior or posterior external surface. A pattern or array of such damage spots can be formed at the depth. In addition, two or more layers of such damage spots can be formed. The depth of the laser focus spot(s) is accurately and rapidly controlled such as to a depth resolution on a micron scale.

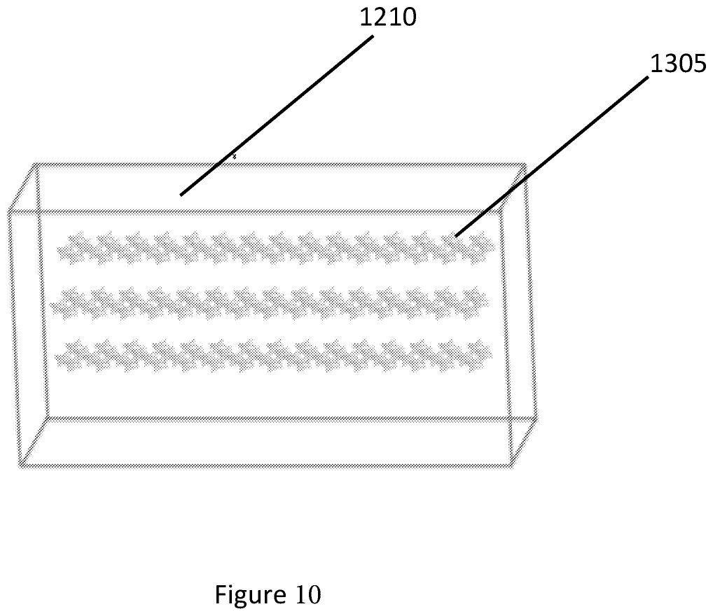

The laser thus forms a two- or three-dimensional array of damage spots that can be arranged in any of a wide variety of patterns. A two-dimensional array includes two or more damage spots positioned in a common plane. A three-dimensional array includes two or more two-dimensional arrays. FIG. 10 shows a schematic representation of a portion of the IOL 1210. It should be appreciated that the portion of the IOL 1210 in FIG. 10 is represented as a prism shape although the shape can vary. A two- or three-dimensional array of damage spots 1305 is positioned entirely below an external surface of the IOL 1210. The array includes one or more damage spots. In the illustrated example, the damage spots form a rectangular-shaped array of equidistant damage spots although the shape and spatial arrangement of the array and the damage spots within the array can vary.

In an example fabrication process for an IOL, the following steps can be performed. First, an IOL is formed such as on a lathe from a plastic (or other material) blank using any well-known process for forming an IOL. The IOL can be machined of any of a variety of materials with an optical zone in the central portion that is configured to enable extended depth of field or monocular focusing. In an embodiment, the IOL is configured having the features described herein with reference to FIGS. 6A-7. Next, the virtual aperture can be formed having flat posterior and anterior surfaces (i.e., the outer surface is not machined or otherwise modified) or the anterior or posterior surfaces can be machined to include desired surface features, such as grooves, ridges, waves, ripples, prisms, or any other surface feature. Next, one or more haptics are machined into the substrate blank according to specifications to allow surgical implantation and proper placement in the eye.

The laser system 1205 is then employed to create a 2 or 3 dimensional pattern of damage spots within the virtual aperture of the IOL as described above.

An alignment process and/or system can be employed to properly align the IOL so that the laser damage is aimed correctly and precisely. The two- or three-dimensional array of damage spots is configured to enable a prescribed or desired amount of light transmission and diffusion thererthrough. For example, the pattern can be a 5-10 layer pattern of 50 micron spots arranged in a rectangular grid or annular grid with 50 micron spacing between damage spots. The pattern can include an offset between layers such that the gaps are filled in when viewed axially. Since a uniform distribution of damage spots can lead to visual artifacts when implanted in an eye, an exemplary spots pattern employs a pseudo-random placement strategy.

In a second example embodiment of a subsurface modification, a femtosecond pulsed laser (FSPL) is configured to interact with the IOL (such as by being focused at a subsurface location of the IOL) to modify a refractive index of one or more subsurface locations of the IOL. The femtosecond pulsed laser forms modified loci in the subsurface locations wherein the modified loci have a different refractive index than the refractive index of the material before modification. Different patterns of modified loci can provide selected dioptic power, toric adjustment, and/or aspheric adjustment provided. The refractive index of the modified loci can also be different from a refractive index at a subsurface location that surrounds the modified loci. The different refractive index may be caused by nonlinear absorption of photons resulting from exposure to focused laser light via the femtosecond pulsed laser.

With reference again to FIG. 9, the laser system 1205 can be configured to emit a femtosecond pulsed laser 1220. The femtosecond laser is focused to a point below the surface of the IOL 1210 and is pulsed in a very specific time and intensity profile. The laser can be controlled in the XY plane, such as by using a Galvo positioning controller, which enables very high speed and high accuracy placement of the beam in the XY plane. Additionally, the system can be coupled to or otherwise use an acoustically controlled focusing mechanism enabling very high frequency and very high accuracy focusing control. This enables positioning of the femtosecond laser focus spot at any depth in the substrate and at any XY coordinate with extreme speed and precision.

The femtosecond laser pulses affect the internal region of the IOL so as to change the refractive index of a specific subsurface region of the IOL and form the loci. The process ca be employed with a wide variety of IOL materials including, for example, glass, hydrophobic and hydrophilic acrylics. The mechanism resulting in the change in refractive index is different in each substrate but in all of the substrates mentioned the laser effected area will have a lower refractive index than the surrounding material. The decrease in refractive index may be dependent on various factors including the specifics of the substrate, the intensity and duration of the laser exposure and the thickness of the material. In general, a change in refractive index of about 0.06 is consistently achievable. For example, if the original hydrophilic acrylic substrate has a nominal refractive index of 1.459 in its fully hydrated configuration, then after laser exposure, the treated areas could have a refractive index as low as 1.399.

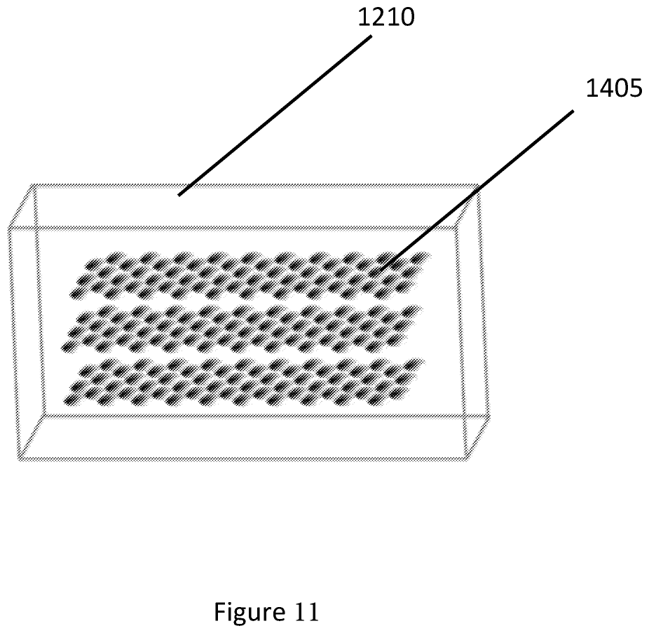

FIG. 11 shows a schematic representation of a portion of the IOL 1210. An array of loci 1405 (each having a modified refractive index) is positioned entirely below an external surface of the IOL 1210. The array includes one or more loci. In the illustrated example, the loci form a rectangular-shaped array of equidistant loci although the shape and spatial arrangement of the array and the loci within the array can vary.

In a sample manufacturing process, the IOL is formed using a lathe to form an IOL with a virtual aperture as described herein. With reference to FIG. 9, the IOL is aligned with a femtosecond laser apparatus 1205, which emits femtosecond laser 1210 focused at a subsurface location to create the a desired subsurface pattern of modified refractive index zones to achieve desired diffusion, transmission, and beam steering.

In any embodiment of subsurface modification, multiple layers of the damage spots or loci within an array permits spreading and homogenization of a narrow beam if light that passes into the IOL.

### Optimization/Enhancement of Optical Zone

As mentioned, the disclosed systems and methods enable a user to manufacture a lens (such as an IOL) having at least one parameter, such as optical zone diameter, a radius of curvature, a conic constant and/or alpha coefficients, that at least partially defines the lens and that maximize or otherwise enhance one or more desired characteristics of the IOL. Some example characteristics or features that may be optimized or enhanced are (1) depth of focus; (2) minimization or reduction of poor (e.g., sub 20/20) performance zones; (3) ensuring robust behavior regarding de-centration, tilt, off axis light sources, surgical implantation error, and errors in choice of IOL Power. The disclosed processes are sometimes referred to as an optimization process. It should be appreciated however that the processes are not limited to optimization but can also be used to enhance or modify the desired characteristics. In an embodiment, a lens such as an IOL is manufactured pursuant to the optimization process.

The virtual aperture, which in any embodiment can be considered a variable transmissivity zone, is configured as described herein and can be modified pursuant to the disclosed optimization procedure. The virtual aperture can be both a scattering mechanism and a partial (or full) light attenuating mechanism. That is, the transmissivity of light for the virtual aperture can be from 0-100%. The optimization procedure essentially includes building or defining of a central optical zone using a standard eye model (such as the equation that defines the lens), wherein parameters of the model are varied using an optimization routine. The parameters are swept through a range of variances to arrive at an optimal optical zone definition as measured by a merit function configured to satisfy predetermined criteria. The eye model can be in the form of an equation that defines an aspect of the lens, such as a surface of the lens. The lens parameter can vary and can be for example, an equation or a part of an equation, such a spheric, aspheric, high order polynomial equation, or any mathematical description of the lens surface where parameter(s) of the equation or formula can be varied. The equation can also be an arbitrary mathematical description with one or more variable parameters that define or more aspects of a lens shape. The lens, such as the lens surface, can also not be defined by any specific formula or equation such that any quantity of locations of the lens surface can be optimized independently. The merit function can vary.

One type of a scattering mechanism in the virtual aperture is a plurality of holes positioned in the virtual aperture. At least some or all of the holes are of predetermined size and positioned on a region or an entirety of the virtual aperture. The holes are configured to permit the passage of light therethrough such as to create diffractive effects and/or to achieve or vary a desired level of opacity. The holes can have a spatial arrangement/pattern, diameter, depth, and/or density of hole patterns that enables a specific light scattering pattern relative to the retina (when implanted in an eye) to achieve desired diffusive effects, such as, for example, homogeneous distribution of light and/or scattering of light.

In an embodiment, the holes are spatially arranged on the virtual aperture to have a density across the surface area of the virtual aperture so as to achieve light transparency in the range of 10 percent to 100 percent light transparency over the entire virtual aperture with the holes being arranged to achieve this. As mentioned, the diameter of the optical zone can vary and can be a minimum of 1.5 mm in a non-limiting example. A 1.5 mm diameter optical zone has been shown to be at or near the minimum threshold required to allow enough light into the eye under mesonic conditions. In another embodiment, the diameter is 1.5 mm to 2.8 mm or 3 mm in size although the diameter can vary and is not limited to a specific size.

In a non-limiting embodiment, the optical zone of the IOL is removable and interchangeable relative to the remainder of the IOL. That is, a structure of the IOL (such as the haptic) other than the optical zone can be implanted and remain in the eye while the optical zone region can be removed and replaced based on prescription or other considerations.

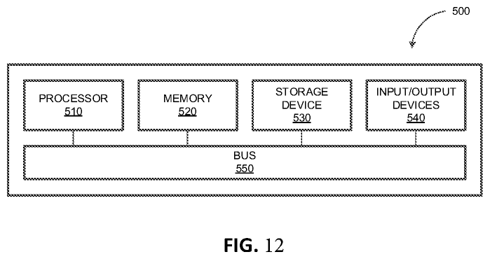

An example optimization or enhancement process is now described. The optimization process can be performed or otherwise implemented using a computing system (such as computing system 500 described below with reference to FIG. 12) wherein the computing system can be directly or indirectly communicatively coupled to a lens manufacturing system. In an initial step, a user selects or identifies a characteristic of the lens to be optimized. Some example characteristics include depth or focus and/or depth of field. For example, the optimization process may select a lens configuration having a depth of field and/or depth of focus each constrained within predetermined end points while maintaining a diameter for the virtual aperture. Other lens characteristics are within the scope of this disclosure. An optimization procedure is then performed that achieves the end points.

In an example method of defining a shape of an ophthalmic lens, a computer and/or user identifies an equation that defines a physical characteristic of the lens, the equation including at least one lens parameter. An output of the equation using a first value for the lens parameter is then achieved, and a merit function is used to score the output. A value of the lens parameter is iteratively varied using an optimization process or optimization algorithm until the merit function achieves an acceptable score. The merit function can be based on visual acuity of the lens and can include at least one of a far visual acuity, a mid visual acuity and a near visual acuity. The merit function can comprise a sum of Log MAR visual acuities for far, mid and near. The merit function can be based on or related to an Airy disc such that an Airy disc value is used as a limit for scoring a focus spot size of the lens. For example, the merit function can compare a spot size to an Airy disc value and provide negative score if the spot size is smaller than or greater than the Airy disc value by a predetermined amount.

The method achieves a lens having sufficient depth of focus to enable a user of the lens to see both distant and near objects with a predetermined visual acuity. The optimization algorithm or function can vary and can include Gradient descent, Newtons method, simulated annealing, Exhaustive Search, brute-force search, stochastic optimization methods, random subsets optimization, greedy or non-greedy algorithms, linear programming optimization, and/or other generate or test techniques. The equation can define a spherical lens, an aspherical lens, or a free form lens surface. The lens parameter can include a radius of curvature and/or a conic constant for example. The physical characteristic can be an outer surface of the lens or other surface of the lens. A lens is manufactured pursuant to the optimization process using any of a variety of lens manufacturing processes.

The optimization process involves sweeping or otherwise providing a range of numerical values corresponding to at least one parameter or feature of the lens (such as an optical zone (wherein the features can include at least radii of curvature and conic constants for example)) and then inserting the numerical values into an equation that describes an aspect or parameter of the lens, such as the following equation (3), which describes an aspheric surface. Equations related to other shapes are within the scope of this disclosure, including spherical and free form optic.

$$z ( r ) = \frac{{r}^{2}}{R ( 1 + \sqrt{1 - ( 1 + K ) \frac{{r}^{2}}{{R}^{2}}} )} + {α}_{4} {r}^{4} + {α}_{6} {r}^{6} + ⋯ , \qquad [ 3 ]$$

where
z(r) is the sag or a z-component of the displacement of the lens surface from a vertex, at distance r from the lens optic axis (lying in a z direction)
r=the radial distance from the axial center of the lens
R=Radius of curvature of the lens
k=Conic constant of the lens
α=coefficient terms (which me be set to 0 in an example although the coefficient can also be optimized in a similar fashion)

A freeform optic is not based on a simple spherical or aspherical profile and the associated mathematic formulae. A freeform optic can take any shape that works to maximize the metrics in the merit function. A freeform optic makes no assumptions about underlying mathematic description such that each point on a lens surface is optimized independently. The agglomeration of all of the points comprise the optical surface.

In an embodiment, numeric values for radius of curvature R and conic constant k are swept across a range of values for the equation (3). For each given radius R and conic constant k, an aspheric lens shape is specified using the equation (3). Next, the lens is assigned a predetermined index of refraction that will be used for a modeling procedure to achieve a plurality of defined lens shapes. Each defined lens shape is positioned in an eye model comprised of a standard accepted eye geometry factors including but not limited to the following eye geometry factors:
Corneal geometry and corneal index of refraction
Aqueous index of refraction
Iris and pupil geometry and placement
Lens placement geometry
Distance to retina
Shape and curvature of retina
Aberrations in the optical system
Known abnormalities of the optical system such as cataracts or posterior capsular opacification (PCO).

Next, the visual acuity for “far” vision” is calculated. In a non-limiting example, “far” distance is a light source at 4000 mm, which represents a clinical standard definition of “far.” This is in Log MAR units where 0 represents 20/20 and smaller values indicate better acuity.

Next, the visual acuity for “near” vision” is calculated. In a non-limiting example, “near” distance is a light source at 400 mm, which represents a clinical standard definition of “near.” Again, this is in Log MAR units where 0 represents 20/20 and smaller values indicate better acuity.

The visual acuity for “mid” vision” is also calculated. In a non-limiting example, “mid” distance is a light source at 660 mm which represents a clinical standard definition of mid. This is in Log MAR units where 0 represents 20/20 and smaller values indicate better acuity.

The above modeled visual acuities are used to find a combination of lens features, such as lens diameter, lens radius of curvature and lens conic constant as defined in equation (3) which has acceptable visual acuity at all three distances.

The correct choice of merit function parameters is important to the optimization process. Any parameter can be included, but the more constrained the merit function becomes, the lower the chances of finding and acceptable solution. In an embodiment, the merit function includes the diameter of optical zone (in the case of the virtual aperture version). The larger the optical zone, the higher the score on the merit function. A larger diameter optical zone is beneficial for overall light gathering ability of the IOL.

The merit function can include a “minimum Log MAR” that is optimized for a particular focus. This indicates the absolute best that an IOL can do for a given pupil size. In an embodiment, an optimal state is to go right to the edge of diffracted effects and then stop “improving” performance. The merit function can also include the variance (or another measure of variability) of the visual acuity between near and far vision. An example optimal in this case is very even performance across the entire focus range without sweet spots.

In an example, the diameter of the optical zone is a minimum of 1.5 mm. A 1.5 mm diameter optical zone has been shown to be at or near the minimum threshold required to allow enough light into the eye under mesonic conditions. In another embodiment, the diameter is 1.5 mm to 2.8 mm or 3 mm in size.

The maximum diameter of the optical zone may limited by the Achievable Visual acuities using the disclosed process. One example criteria is that there is always at least one distance (far, mid or near that achieves a Log MAR of 0 or lower thus 20/20 visual acuity or better). Another example criteria is that the mid distance vision be 20/20 or better while maintaining acceptable near and far vision. As the diameter of the optical zone gets bigger, this may become more difficult to achieve.

An example of this is shown in the table below. The first row represents a 3 mm diameter optic with 17.7 mm radius of curvature and k=−100. In this example, the mid visual acuity is better than 20/20 and both near and far are close to 20/20. The second row shows a 4 mm diameter optic where none of the distances achieve 20/20 visual acuity.

Standard mathematic optimization methods or algorithms are then used to compute combinations of parameters that achieve desired results. Some example optimization techniques include but are not limited to: Gradient descent, Newtons method, Simulated annealing, Exhaustive Search, brute-force search, stochastic optimization, random subsets optimization, greedy or non-greedy algorithm, linear programming optimization, and/or generate and test techniques. The merit function used in the optimization is flexible, but an example is the sum of the Log MAR visual acuities for far, mid and near. The sum represents an overall

| radius | K | diopters | sfl | Far VA |
|---|---|---|---|---|
| 3.0 mm Diameter | | | | |
| 17.7 | −100 | 21.773015353737300 | 20.122053649052300 | 0.02177975562212580 |
| 4.0 mm Diameter | | | | |
| 17.7 | −100 | 21.773015353737300 | 20.122053649052300 | 0.042995636220620800 |

| radius | mid VA | Near VA | Sum VA |
|---|---|---|---|
| 3.0 mm Diameter | | | |
| 17.7 | −0.006902535414526210 | 0.05696184203916190 | 0.0718390622467615 |
| 4.0 mm Diameter | | | |
| 17.7 | 0.01080676643497880 | 0.07064840389873430 | 0.124450806554334 |

performance metric as lower is better in all three distances. In the example above, the 3 mm optic has a sum of 0.0718 while the 4.0 mm optic has a sum of 0.1244. In this case the 3 mm optic is superior and would rank higher in the optimization. Another example merit function can include the difference between the near and Log MAR values which would indicate the far near power balance of the lens.

In an embodiment, the lens optimization procedure achieves a lens that can be manufactured having sufficient depth of focus to enable a user of the lens to see both distant and near objects with good visual acuity. The optimizer utilizes constraints of distant, mid, and near models and predicts the visual acuity at all three constraints. One or more parameters, such as radius of curvature and conic constant, are varied until both distant, near and mid visual acuity perform as desired. Additionally, the alpha coefficients in the equation can also be optimized to further enhance performance.

A lens can be manufactured pursuant to the optimization processes described herein and implanted into a person's eye.

FIG. 12 depicts a block diagram illustrating an example of a computing system 500 consistent with implementations of the current subject matter. The computing system 500 may implement any of the process described herein. As shown in FIG. 12, the computing system 500 can include a processor 510, a memory 520, a storage device 530, and input/output device 540. The processor 510, the memory 520, the storage device 530, and the input/output device 540 can be interconnected via a system bus 550. The processor 510 is capable of processing instructions for execution within the computing system 500. Such executed instructions can implement one or more components of the processes described herein. In some implementations of the current subject matter, the processor 510 can be a single-threaded processor. Alternately, the processor 510 can be a multi-threaded processor. The processor 510 is capable of processing instructions stored in the memory 520 and/or on the storage device 530 to display graphical information for a user interface provided via the input/output device 540.

The memory 520 is a computer readable medium such as volatile or non-volatile that stores information within the computing system 500. The memory 520 can store data structures representing configuration object databases, for example. The storage device 530 is capable of providing persistent storage for the computing system 500. The storage device 530 can be a floppy disk device, a digital cloud, a hard disk device, an optical disk device, or a tape device, or other suitable persistent storage means. The input/output device 540 provides input/output operations for the computing system 500. In some implementations of the current subject matter, the input/output device 540 includes a keyboard and/or pointing device. In various implementations, the input/output device 540 includes a display unit for displaying graphical user interfaces.

According to some implementations of the current subject matter, the input/output device 540 can provide input/output operations for a network device. For example, the input/output device 540 can include Ethernet ports or other networking ports to communicate with one or more wired and/or wireless networks, Bluetooth or digital cloud system (e.g., a local area network (LAN), a wide area network (WAN), the Internet).

In some implementations of the current subject matter, the computing system 500 can be used to execute various interactive computer software applications that can be used for organization, analysis and/or storage of data in various (e.g., tabular) format (e.g., Microsoft Excel®, and/or any other type of software). Alternatively, the computing system 500 can be used to execute any type of software applications. These applications can be used to perform various functionalities, e.g., planning functionalities (e.g., generating, managing, editing of spreadsheet documents, word processing documents, and/or any other objects, etc.), computing functionalities, communications functionalities, etc. The applications can include various add-in functionalities, plug ins, or can be standalone computing products and/or functionalities. Upon activation within the applications, the functionalities can be used to generate the user interface provided via the input/output device 540. The user interface can be generated and presented to a user by the computing system 500 (e.g., on a computer screen monitor, etc.). The user interface can be integrated with other devices or virtual ecosystems.

While this specification contains many specifics, these should not be construed as limitations on the scope of an invention that is claimed or of what may be claimed, but rather as descriptions of features specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination. Similarly, while operations are depicted in the drawings in a particular order, this should not be understood as requiring that such operations be performed in the particular order shown or in sequential order, or that all illustrated operations be performed, to achieve desirable results. Only a few examples and implementations are disclosed. Variations, modifications and enhancements to the described examples and implementations and other implementations may be made based on what is disclosed.

1. A method of defining a shape of an ophthalmic lens, comprising:
identifying an equation that defines a physical characteristic of the lens, the equation including at least one lens parameter;
achieving an output of the equation using a first value for the lens parameter;
using a merit function to score the output;
iteratively varying a value of then at least one lens parameter using an optimization algorithm until the merit function achieves an acceptable score.

2. The method of claim 1, wherein the merit function is based on visual acuity of the lens.

3. The method of claim 2, wherein the merit function includes at least one of a far visual acuity, a mid visual acuity and a near visual acuity.

4. The method of claim 2, wherein the merit function comprises a sum of Log MAR visual acuities for far, mid and near.

5. The method of claim 1, wherein method achieves a lens having sufficient depth of focus to enable a user of the lens to see both distant and near objects with a predetermined visual acuity.

6. The method of claim 1, wherein the optimization algorithm includes Gradient descent, Newtons method, simulated annealing, exhaustive Search, brute-force search, stochastic optimization methods, random subsets optimization, greedy or non-greedy algorithms, linear programming optimization, and/or generate or test techniques.

7. The method of claim 1, wherein the equation defines a spherical lens.

8. The method of claim 1, wherein the equation defines an aspherical lens.

9. The method of claim 1, wherein the equation is a high order polynomial equation that defines a surface of the lens.

10. The method of claim 1, wherein the equation defines a free form lens surface.

11. The method of claim 1, wherein the at least one lens parameter is a radius of curvature.

12. The method of claim 1, wherein the at least one lens parameter is conic constant.

13. The method of claim 1, wherein the physical characteristic is an outer surface of the lens.

14. The method of claim 1, further comprising manufacturing the lens.
